(19) United States
(12) Patent Application Publication
(10) Pub. No.: US 2026/0215690 A1
(43) Pub. Date: Jul. 30, 2026

(54) METHODS AND SYSTEMS FOR DETERMINING MEASURES OF PHYSIOLOGICAL RESPONSE DUE TO HEAT

(71) Applicant: greenteg AG, Rumlang (CH)

(72) Inventors: Tobias Schmid, Pfyn (CH); Michele Zahner, Zurich (CH); Marcel Ott, Wallisellen (CH); Wulf Glatz, Zurich (CH); Lukas Durrer, Rickenbach SZ (CH); Chris Blomfield-Brown, Thun (CH)

(21) Appl. No.: 19/141,808

(22) PCT Filed: Dec. 12, 2023
(86) PCT No.: PCT/EP2023/085394

§ 371 Date: Jun. 20, 2025

(30) Foreign Application Priority Data
Dec. 23, 2022 (CH) CH001588/2022

(51) Int. Cl. A61B 5/01; A61B 5/00; A61B 5/0205; A61B 5/024; A61B 5/0245; A61B 5/0533; A61B 5/08; A61B 5/145

(52) U.S. Cl. CPC A61B 5/01; A61B 5/02055; A61B 5/02405; A61B 5/02416; A61B 5/0245; A61B 5/0533; A61B 5/0816; A61B 5/14542; A61B 5/14546; A61B 5/4266; A61B 5/4875; A61B 5/6823; A61B 5/6824; A61B 5/6831; A61B 5/7282; A61B 5/746; A61B 2560/0242; A61B 2560/0462; A61B 2562/0219; A61B 2562/0271

(57) ABSTRACT

A computer-implemented method and an electronic system for determining a measure of the physiological strain on a mammal due to heat are disclosed, wherein a number of steps are performed: receiving (S13) a measurement of a core body temperature (CBT) and a skin temperature (ST) of the mammal; calculating (S14) a mean body temperature 5 (MBT) as a function of the core body temperature (CBT) and the skin temperature (ST); and calculating (S15) a heat strain index (HSI) as a function of the mean body temperature (MBT), the heat strain index (HSI) indicative of the physiological strain on the mammal due to heat.

### FIELD OF THE DISCLOSURE

The present disclosure relates to methods and systems for determining measures of a physiological response on a mammal, in particular a human, due to heat.

### BACKGROUND OF THE DISCLOSURE

Aerobic exercise performance is degraded by heat stress when studied in both in laboratories and field settings. Many factors contribute to the heat stress to which a human body is subjected: metabolic heat (which can increase during work or exercise), environmental factors (e.g., thermal radiation (e.g., from exposure to the Sun), ambient temperature, ambient humidity), and clothing. Heat stress in mild or moderate doses can cause discomfort and may adversely affect physical and mental performance, but is not harmful to health. As the heat stress increases and approaches human tolerance limits, the risks of heat-related disorders increases, such as heat syncope, heat cramps, and heat exhaustion.

While improving athletic performances in hot conditions has been the subject of much research, measuring and quantifying the amount of heat a human is subject to also plays a crucial role for worker health and safety, not only in industrial settings such as thermal power plants, forges, or other industrial processes which generate large amounts of heat, but agricultural settings where workers can spend long hours under the sun.

Not only humans experience heat stress, but animals also. In particular, mammals, including pets such as dogs and cats, and farm animals including cows, horses, etc. experience heat stress and research has been performed on the impacts of heat stress. According to Godde et al. (2021), “the effects of heat stress include reduced productivity, reduced animal welfare, reduced fertility, increased susceptibility to disease, and in extreme cases increased mortality and affect all domesticated species.”

Heat strain is the overall physiological response of an individual person resulting from heat stress, and can vary from person to person. The body responds by a process called thermoregulation, for example involving sweating, which is dedicated to dissipating excess heat from the body. Thermoregulation uses physiological resources of the body and therefore leads to less resources being available for other tasks (e.g., mental and physical performance).

In the present disclosure, the term heat strain includes overall changes in the mammal due to heat, in particular also the increase in temperature of the body (e.g., the skin temperature and/or the core temperature). The increase in heat can be due to internal processes related to exercise and/or due to external influences.

With regular exposure to heat stress, the body's tolerance increases and therefore the heat strain reduces (Tyler, Reeve, Hodges, & Cheung, 2016). This process is called acclimatization. To achieve a high degree of acclimatization, physical activity under heat stress conditions similar to those anticipated during work (e.g. during sports or physical labour) is required. According to the prior art, for example, a person with a recent history of heat stress exposures of at least two continuous hours (e.g., 5 of the last 7 days to 10 of the last 14 days), can be considered acclimatized. The body acclimatizes, for example, by increasing the sweating efficiency (earlier onset of sweating, greater sweat production, and reduced electrolyte loss in sweat), increased vasodilation, reduced core and skin temperatures, as well as improved fluid balance and cardiovascular stability. The level of acclimatization depends on the type of induced heat-stress (i.e., passive vs. exercise-induced heat stress, dry vs. humid heat exposure), intensity, duration, frequency, and number of heat exposures (Taylor, 2014; Periard et al., 2015). If a person is not exposed to heat stress for some time, the degree of heat acclimatization lessens.

The core body temperature of a person is an important factor for determining a person's thermal state and is a widely accepted surrogate for estimating heat stress. However, according to some studies, the core body temperature alone does not suffice to estimate the reduction in athletic performance due to heat stress. Cheuvront et. al (2003) have shown that at a given core body temperature, the exhaustion point is reached earlier with a higher skin temperature due to an increase in heart rate and blood flow to the skin across the whole body. A smaller temperature gradient between the skin and the core forces the body to increase the blood flow to the skin to reach the same heat transport. This affects the available blood for the working muscles and impairs the performance (González-Alonso, et al., 1999).

WO2019108699A1 discloses a system or a method for assisting an individual to acclimatize to a hot environment prior to being exposed to the hot environment using a measured heart rate with or without a measured skin temperature and/or a measured body core temperature. A Physiological Strain Index (PSI) or adaptive PSI (aPSI) is calculated for the individual to provide a target for the individual's exertion level such that an area under the calculated PSI/aPSI curve is used to determine the amount of heat acclimatization that has occurred for that particular training session and/or prior training sessions. The disadvantage of the teaching of this publication is that the core body temperature as calculated using only the heart rate does not involve a direct measurement of a temperature and therefore is based on assumptions which are not particularly reliable in all scenarios, for example as it does not take into sufficient account differences between individuals or all exercise conditions. Though the publication does mention the use of an internal temperature sensor which wirelessly transmits a body core temperature for the individual, swallowing a sensor is cumbersome and not suited for regular (in particular daily) use. Further, determining the physiological strain index requires the use of a heart rate monitor. Additionally, using the skin temperature itself is also error prone as the skin temperature varies depending on the location on the body where the temperature is placed.

### SUMMARY OF THE DISCLOSURE

It is an object of the disclosure and embodiments disclosed herein to provide methods and systems for determining measures of a physiological response on a mammal, in particular a human, due to heat.

In particular, it is an object of the disclosure and embodiments disclosed herein to provide a computer-implemented method and an electronic system for determining a measure of the physiological strain on a mammal, in particular a human, due to heat which does not have at least some disadvantages of the prior art.

In addition, it is an object of the disclosure and embodiments disclosed herein to provide a computer-implemented method and an electronic system for determining a measure of the physiological load on a mammal, in particular a human, due to heat which does not have at least some disadvantages of the prior art.

In addition, it is an object of the disclosure and embodiments disclosed herein to provide a computer-implemented method and an electronic system for determining a measure of an acclimatization adjusted physiological strain on a mammal, in particular a human, due to heat which does not have at least some disadvantages of the prior art.

In addition, it is an object of the disclosure and embodiments disclosed herein to provide a computer-implemented method and an electronic system for determining a measure of the readiness of a mammal, in particular a human, to perform under heat stress, which does not have at least some disadvantages of the prior art.

At least some of the steps described with reference to one of the objects of this disclosure, in particular a first method, may also be performed with reference to another of the objects of this disclosure, in particular a second method.

The present disclosure relates to a computer-implemented method for determining a measure of the physiological strain on a mammal due to heat. The method comprises receiving a measurement of a core body temperature and a skin temperature of the mammal. The method comprises calculating a mean body temperature as a function of the core body temperature and the skin temperature. The method comprises calculating a heat strain index as a function of the mean body temperature, the heat strain index indicative of the physiological strain on the mammal due to heat.

In an embodiment, the heat strain index is calculated using only the mean body temperature. In particular, the heat strain index is calculated without using heart rate.

In an embodiment, the mammal is a human.

In an embodiment, the mammal is a domesticated mammal, for example a dog, cat, cow, horse, donkey, etc.

In an embodiment, the measurement of the core body temperature and the skin temperature is taken at a defined position on the human body, in particular a single defined position. The method comprises correcting the skin temperature using a defined correction term associated with the defined position. For example, the defined correction term comprises an offset value of 0.3° C.-1° C.

In an embodiment, the defined position is the torso, in particular a chest area, more particularly a left side of the chest between the left armpit and the left hip. The defined correction term comprises an offset value which is, for example, 0.4° C.-0.8° C., preferably 0.5° C.

In an embodiment, the defined position is the arm, preferably the upper arm and/or the wrist. The defined correction term comprises an offset value which is, for example, 0.6° C.-1.0° C., preferably 0.7° C.

In an embodiment, the defined position is selected from one of the following positions: the torso, the upper arm, or the wrist. The defined correction term is an individual correction term, determined for the particular human. The defined correction term can take into account clothing.

The defined correction term relates more specifically to the distribution in heat transfer coefficient between the skin and the environment across the skin surface.

In an embodiment, the measurement of the core body temperature and the skin temperature is taken at a plurality of the defined positions described herein.

In an embodiment, the measurement of the core body temperature is taken at a different defined position than the skin temperature.

In an embodiment, the skin temperature measurement comprises a plurality of skin temperature measurements taken at different defined positions.

In an embodiment, the defined correction term associated with the defined position is determined by a number of steps. The steps include measuring the skin temperature at a plurality of different positions on the body, preferably at least three, including the defined position. The steps include calculating a mean skin temperature using the plurality of measurement. The steps include determining the defined correction term using a difference between the mean skin temperature and the skin temperature at the defined position.

In an embodiment, the method comprises receiving one or more further physiological signals of the mammal, including heart rate, heart rate variability, galvanic skin response, sweat rate, breathing rate, oxygen saturation, heat flux, movement data, glucose concentration, and/or lactate concentration. The method comprises calculating the heat strain index further using the one or more further physiological signals.

In an embodiment, the method comprising calculating the heat strain index as a function of the mean body temperature, a lower temperature threshold and an upper temperature threshold.

In an embodiment, the lower temperature threshold is 36° C.-37° C., preferably 36.7° C. The upper temperature threshold is 38° C.-40° C., preferably 38.9° C.

In an embodiment, the method comprises calculating the core body temperature as a function of a heat flux (HF) at the skin and the skin temperature (ST) at the defined location.

In an embodiment, the core body temperature is calculated as a function of a PPG signal.

In an embodiment, the core body temperature is calculated as a function of heart rate and at least one skin temperature.

In an embodiment, the core body temperature is calculated as a function of at least one skin temperature and one ambient temperature.

In an embodiment, the core body temperature is calculated as a function of at least one skin temperature, at least one ambient temperature, and heart rate.

In an embodiment, the core body temperature is calculated as a function of at least one heat flux sensor, at least one skin temperature sensor, and heart rate.

In an embodiment, the core body temperature is calculated as a function of at least one skin temperature sensor and one ambient temperature sensor.

In an embodiment, the core body temperature is calculated as a function of an accelerometer reading, a galvanic skin response, a sweat rate, and/or a blood oxygen saturation.

In an embodiment, the core body temperature is calculated as a function of one or more of: PPG, ECG (including heartrate and/or heartrate variability), skin temperature, ambient temperature, device temperature, accelerometer, galvanic skin response, sweat rate, blood oxygen saturation, breathing rate and/or blood pressure.

In an embodiment, the mean body temperature is calculated as a linear combination of the core body temperature and the skin temperature, preferably a weighted sum comprising a core body temperature coefficient and a skin temperature coefficient.

In an embodiment, the mean body temperature is calculated as a normalized weighted sum where the core body temperature has a coefficient of 0.6-0.7, preferably 0.64, and where the skin temperature has a coefficient of 0.3-0.4, preferably 0.36.

In an embodiment, the heat strain index is proportional to a difference between the mean body temperature and the lower temperature threshold and inversely proportional to a difference between the upper temperature threshold and the lower temperature threshold.

The present disclosure also relates to a computer-implemented method for determining a measure of the physiological load placed on a mammal, in particular a human, due to heat. The method comprises calculating a heat strain index as a function of a body temperature, preferably as described herein, of the mammal, in particular a mean body temperature, preferably as described herein. The method further comprises determining a heat strain score indicative of a cumulative heat load for a period of elevated heat or part thereof, the heat strain score determined using an average and/or a normalized heat strain index and a duration of the period of elevated heat or part thereof.

In an embodiment, the cumulative heat load is calculated as a function of an integral of the heat strain index for a period of elevated heat or part thereof.

In an embodiment, the methods comprises determining the average heat strain index for a period of elevated heat or a part thereof as a mean of a plurality of heat strain index values.

In an embodiment, the method further comprises determining the normalized heat strain index for a period of elevated heat or a part thereof by calculating an i-th power of each of a plurality of heat strain index values, calculating a mean value of the i-th powers, and calculating the i-th root of the mean value, wherein i is a number (a real number) between 2-5.

In an embodiment, the method further comprises generating an acute heat load with reference to a particular time-point as a weighted sum of one or more heat strain scores associated with one or more past periods of elevated heat, wherein the weight of a given heat strain score depends on a duration between the particular time-point and the date of the given past period of elevated heat, wherein the weight decreases as the duration increases.

In an embodiment, the weight of a given heat strain score, when calculating the acute heat load, is subject to exponential decay with a time constant of 2-10 days.

In an embodiment, the method further comprises generating a chronic heat load with reference to a particular time-point as a weighted sum of one or more heat strain scores associated with one or more past periods of elevated heat, wherein the weight of a given heat strain score depends on a duration between the particular time-point and the date of the given past period of elevated heat, wherein the weight decreases as the duration increases, wherein the decrease is less rapid than a decrease of the weights used for generating the acute heat load.

In an embodiment, the weight of a given heat strain score, when computing a chronic heat load, is subject to exponential decay with a time constant of 5-30 days.

The present disclosure also relates to a method for determining a measure of the readiness of a mammal, in particular a human, to experience elevated heat. The method comprises receiving a plurality of heat strain scores of past activities, the heat strain score preferably determined as described herein. The method further comprises determining a chronic heat load, the chronic heat load preferably determined as described herein. The method further comprises determining an acute heat load, the acute heat load preferably determined as described herein. The method comprises determining a heat readiness index as a function of the chronic heat load and the acute heat load, preferably as a difference between the chronic heat load and the acute heat load.

In an embodiment, the method further comprises generating an alarm signal (or alternatively, an alert signal or a warning signal) if the heat strain index exceeds a heat strain index threshold and/or if the heat strain score exceeds a heat strain score threshold.

In an embodiment, the heat strain threshold is a generic threshold suitable for all mammals/humans. Alternatively or additionally, the heat strain threshold is specific either to a group of individuals, or to a particular individual. The groups of individuals may include occupational groups, climate zone groups, age groups, sports specific groups, etc.

In an embodiment, the method further comprises generating a message including the heat strain index. Optionally, the method includes displaying, on a display, the heat strain index. Optionally, the method includes displaying, on the display, the heat strain score and/or the alarm message.

The present disclosure also relates to a method for determining a measure of an acclimatization adjusted physiological strain on a mammal, in particular a human, due to heat. The method comprises receiving a body temperature, preferably a mean body temperature as described herein. The method comprises determining a heat strain index using the body temperature, preferably using a method as described herein. The method comprises determining an acclimatization adjusted heat strain index. The acclimatization adjusted heat strain index is determined using the heat strain index and one or more further physiological signals of the mammal including: heart rate, heart rate variability, galvanic skin response, hydration status, body weight, sweat rate, breathing rate, oxygen saturation, heat flux, movement data, performance data, glucose concentration, and/or lactate concentration.

In an embodiment, the method further comprises determining an acclimatization index, indicative of a degree of acclimatization of the person, the acclimatization index determined as a function of the acclimatization adjusted heat strain index and the heat strain index.

In addition to a computer-implemented method for determining a measure of the physiological strain on a mammal due to heat, the present disclosure also relates to an electronic system for performing one or more of the methods described herein. The electronic system comprises a processor configured to perform one or more of the computer-implemented methods as described herein.

In an embodiment, the electronic system further comprises a wearable device. The wearable device includes a sensor system configured to determine the core body temperature and the skin temperature of the mammal, wherein the sensor system is worn on the body of the mammal.

In an embodiment where the mammal is a human, the sensor system is worn in skin contact on the body of the human.

In an embodiment, the sensor system comprises a heat flux sensor configured to determine the core body temperature and a temperature sensor configured to determine the skin temperature. In particular, the heat flux sensor comprises a series of p- and n-doped semiconductors.

In an embodiment, the electronic system comprises the following sensors: a PPG sensor, an ECG sensor, a blood lactate sensor, a galvanic skin response sensor, a sweat rate sensor, one or more further skin temperature sensors, an ambient temperature sensor, and/or an accelerometer. Preferably, the additional sensors are integrated into the wearable device.

In an embodiment, the wearable device comprises a strap for securing the wearable device to the body of a human. The strap is preferably a chest strap or a wrist strap.

In an embodiment, the wearable device is worn on the torso, in particular a chest area, more particularly a left side of the chest between the left armpit and the left hip (apical position).

In an embodiment, the processor is integrated into the wearable device.

In an embodiment, the wearable device includes a wireless communications module configured to generate and transmit a message comprising the current heat strain index. The heat strain index is, for example, transmitted to one or more user devices, such as a smart watch, mobile phone, and/or sports computer (i.e. a running watch, cycling computer, rowing computer, etc.)

In an embodiment, the electronic system further comprises a separate electronic device. The processor is integrated into the separate electronic device. The wearable device includes a wireless communications module configured to transmit a message including the core body temperature and the skin temperature to the separate electronic device. The separate electronic device can be a user device as described herein. The separate electronic device could also be a server computer located remotely.

In addition to computer-implemented methods and an electronic system for implementing the computer-implemented methods, the present disclosure also relates to a computer program product for performing one or more of the methods described herein. The computer program product comprises computer program code configured to control a processor such that the processor performs one or more of the methods described herein.

The present disclosure also relates to a non-transitory computer readable medium having stored thereon computer program code configured to control a processor such that the processor performs one or more of the methods described herein.

The embodiments described above apply to one or more of the methods herein. In particular, the features of a specific embodiment are not limited to the particular method with which they may be construed to relate to, rather, the skilled person knows that the teaching of the specific embodiment may also be applied in another method.

### BRIEF DESCRIPTION OF THE DRAWINGS

The herein described disclosure will be more fully understood from the detailed description given herein below and the accompanying drawings, which should not be considered limiting to the invention described in the appended claims. The drawings in which:

FIG. 1 shows a diagram illustrating a human and indicating several positions on the human body where a wearable device as described herein may be worn;

FIG. 2 shows schematically a front view of a wearable device comprising a sensor system;

FIG. 3 shows schematically a side view of a wearable device comprising a sensor system and indicating a heat flow through the sensor system;

FIG. 4 shows a block diagram illustrating schematically an electronic system comprising a wearable device and a processor;

FIG. 5 shows a block diagram illustrating schematically an electronic system comprising a wearable device having an integrated processor;

FIG. 6 shows a diagram illustrating schematically an electronic system including a wearable device connected to a user device, the user device including a processor;

FIG. 7 shows a diagram illustrating schematically an electronic system including a wearable device, a server computer, and a user device;

FIG. 8 shows a diagram illustrating schematically an electronic system including a wearable device with a display and a server computer;

FIG. 9 shows a block diagram illustrating schematically a sensor system comprising a heat flux sensor and a skin temperature sensor;

FIG. 10 shows a block diagram illustrating schematically a sensor system comprising a heat flux sensor, a skin temperature sensor, and optional further sensors;

FIG. 11 shows a flow diagram illustrating a method for determining a measure of the physiological strain on a mammal due to heat, in particular for determining a heat strain index;

FIG. 12 shows a flow diagram illustrating a method for determining a measure of a physiological strain on a mammal due to prolonged heat exposure;

FIG. 13 shows a flow diagram illustrating a method for determining a measure of the acclimatization adjusted physiological strain as a result of heat;

FIG. 14 shows a flow diagram illustrating a method for determining a measure of the readiness of a mammal to experience elevated heat;

FIG. 15 shows a flow diagram illustrating a method for determining a defined correction term for correcting a skin temperature measurement;

FIG. 16 shows a time-series chart of the measured core body temperature, the measured skin temperature, and the calculated mean body temperature during a session of low intensity indoor cycling; and

FIG. 17 shows a time-series chart of the measured core body temperature, the measured skin temperature, and the calculated mean body temperature during two high intensity cycling sessions, a first outdoor session and a second indoor session.

### DESCRIPTION OF THE EMBODIMENTS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

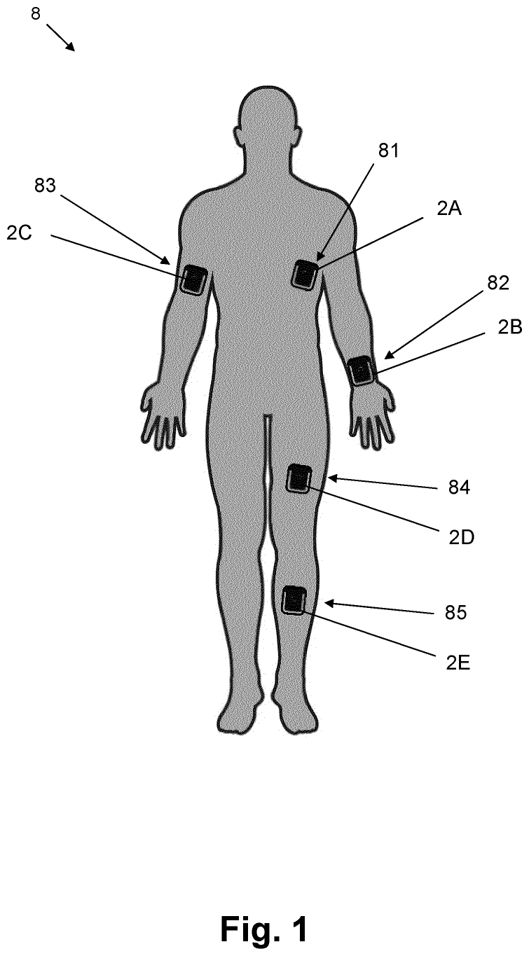

FIG. 1 shows a human body 8. Illustrated are a number of possible positions 81, 82, 83, 84, 85 on the body 8 where a wearable device 2A, 2B, 2C, 2D, 2E can be worn, however other positions are possible. Typically, a single wearable device 2A, 2B, 2C, 2D, 2E is worn at one of the positions 81, 82, 83, 84, 85. The positions 81, 82, 83, 84, 85 include a chest area 81, in particular on the left chest area (the so-called apical region), a wrist region 82 (in particular on a dorsal side of the wrist), an upper arm 83, an upper leg 84, and a lower leg 85. Other possible positions include the head (e.g., the forehead or a temple region), forearm, ankle, finger, fingertip, earlobe, or inside the ear.

The wearable device 2A, 2B, 2C, 2D, 2E is preferably worn in direct body contact (in particular, direct skin contact) for best results. However, the wearable device 2A, 2B, 2C, 2D, 2E may also be worn over one or more layers of clothing, or over hair or fur (in the case of mammals other than humans).

Depending on the embodiment, the wearable device 2A, 2B, 2C, 2D, 2E is implemented as a single device, or implemented as a distributed device with one or more connected parts.

In an embodiment where the wearable device 2A, 2B, 2C, 2D, 2E is worn on a limb of the body 8 or worn on the torso, the wearable device 2A, 2B, 2C, 2D, 2E is typically affixed to the body 8 by means of a strap or band. The wearable device 2A, 2B, 2C, 2D, 2E may also be at least partially integrated into a garment, in particular a garment designed for being worn in skin contact.

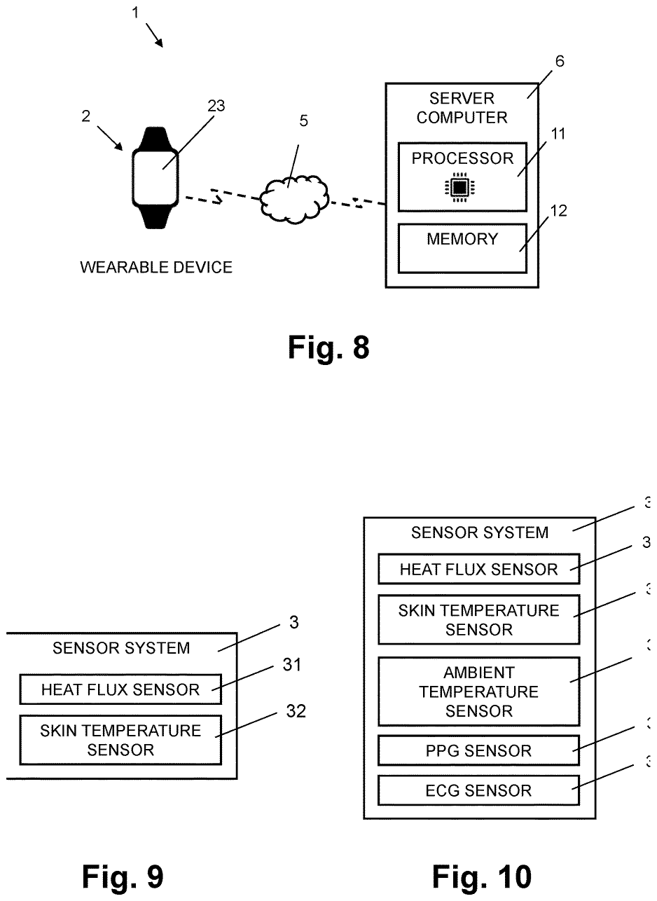

In an embodiment where the wearable device 2A, 2B, 2C, 2D, 2E is configured to be worn on the wrist, the wearable device 2A, 2B, 2C, 2D, 2E may be implemented in the form of an electronic bracelet, electronic cuff, or electronic watch, for example. In particular, the wearable device 2A, 2B, 2C, 2D, 2E can be implemented as a health tracker, fitness tracker, and/or a smart watch (as is depicted in FIG. 8, for example).

In an embodiment where the wearable device 2A, 2B, 2C, 2D, 2E is configured to be worn on the head, in particular on a forehead or a temple region, the wearable device 2A, 2B, 2C, 2D, 2E may be implemented as an electronic headband, electronic glasses (i.e., smart glasses), an electronic hat, or an electronic helmet, for example.

As is explained below, the wearable device 2A, 2B, 2C, 2D, 2E includes a sensor system (not shown). The sensor system may include one or more sensors directly integrated into the wearable device 2A, 2B, 2C, 2D, 2E (i.e. having a housing in common with the 2A, 2B, 2C, 2D, 2E). However, the wearable device 2A, 2B, 2C, 2D, 2E may also include one or more auxiliary sensors which are not directly integrated into the wearable device 2A, 2B, 2C, 2D, 2E but are connected to the wearable device 2A, 2B, 2C, 2D, 2E using a wired or wireless data communication system.

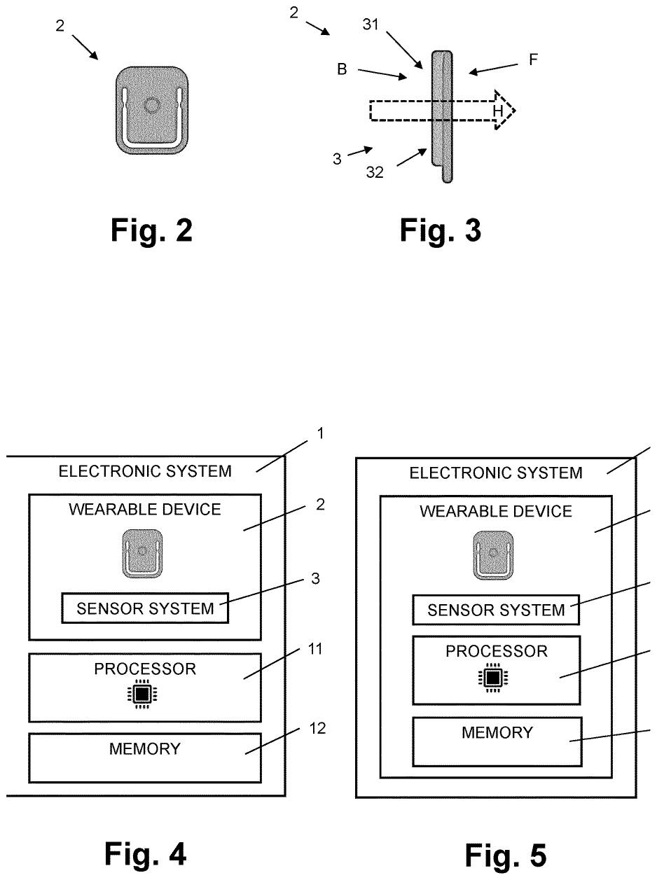

As shown in FIGS. 2 and 3, the wearable device 2 has a substantially rectangular housing having a front side F and a back side B. The wearable device 2 is thin in comparison to its lateral dimensions. The wearable device 2 comprises a sensor system 3, in particular including a core body temperature sensor and a skin temperature sensor.

The core body temperature sensor may be implemented using a heat flux sensor 31 and a skin temperature sensor 32 as is explained in more detail with reference to FIG. 9. The sensor system 3 can comprise further sensors as is explained in more detail with reference to FIG. 10.

When worn on a body 8, the back side B faces the body, preferably in direct skin contact with the body 8. The arrow indicates the heat flux H which flows from the body 8 through the wearable device 2 from the back side B to the front side F.

FIGS. 4-8 show examples of an electronic system 1. The electronic system 1 comprises at least one processor 11 configured to carry out one or more steps and/or functions as described herein.

Depending on its configuration, the electronic system 1 further includes various components, such as a memory 12, a communication interface, and/or a user interface. The components of the electronic system 1 are connected to each other via a data communication system, such that they can transmit and/or receive data.

The term data communication system relates to a communication system that facilitates data communication between two components, devices, systems, or other entities. Depending on its configuration, the data communication system is wired and includes a wired connection, such as a cable and/or a system bus, and/or includes a wireless connection, such as Bluetooth (BT), Bluetooth Low Energy (BLE), ANT+, WiFi, RFID, etc. The data communication system may further include communication modules for communication via networks, such as local area networks (LANs), mobile radio networks (e.g., GSM, GPRS, CDMA2000, EDGE, and/or UTMS), and/or the Internet 5. The Internet 5 includes, depending on the implementation, intermediary networks.

The processor 11 may comprise a system on a chip (SoC), a central processing unit (CPU), and/or other more specific processing units such as a graphical processing unit (GPU), application specific integrated circuits (ASICs), or reprogrammable processing units such as field programmable gate arrays (FPGAs).

The memory 12 comprises one or more volatile (transient) and/or non-volatile (non-transient) storage components. The storage components may be removable and/or nonremovable, and can also be integrated, in whole or in part with the processor 11. Examples of storage components include RAM (Random Access Memory), flash memory, hard disks, data memory, and/or other data stores. The memory 12 comprises a non-transitory computer-readable medium having stored thereon computer program code configured to control the processor 11, such that the electronic system 1 performs one or more steps and/or functions as described herein. Depending on the embodiment, the computer program code is compiled or non-compiled program logic and/or machine code. As such, the electronic system 1 is configured to perform one or more steps and/or functions.

The computer program code defines and/or is part of a discrete software application. One skilled in the art will understand that the computer program code can also be distributed across a plurality of software applications (Apps). In an embodiment, the computer program code further provides interfaces, such as APIs, such that functionality and/or data of the electronic system 1 can be accessed remotely, such as via a client application or via a web browser.

While particular steps and/or functions are described herein as being performed by a particular component or device of the electronic system 1, particular steps and/or functions may be performed in other components or devices of the electronic system 1 in whole or in part. Further, particular steps disclosed as being performed by the processor 11 may be performed by the wearable device 2. Additionally or alternatively, particular steps as disclosed as being performed by the processor 11 may be performed by a plurality of processors 11, in particular a plurality of processors 11 distributed across one or more devices of the electronic system 3.

As depicted in FIGS. 4-8, the electronic system 1 comprises the wearable device 2. The wearable device includes the sensor system 3.

The wearable device 2 may include a communications module configured for data communication with other devices, in particular with other devices of the electronic system 1, using the data communication system. Depending on the embodiment, the communications module may be configured for wired and/or wireless communication.

Depending on the embodiment, the wearable device 2 may also include further electronic components, in particular a power source such as a battery. Other electronic components include, for example, a user interface configured to receive user input and/or provide information to the user, for example comprising user input means such as a touch screen, buttons, a rotary wheel, etc. The user interface may provide information to the user by means of a display (e.g., a screen, a touch screen, and/or an AR display system), a loudspeaker, or haptic feedback (e.g. using vibrations).

In an embodiment, the wearable device 2 further comprises modules configured to determine a current time, an orientation of the wearable device 2, a location of the wearable device 2 (e.g. using a GNSS receiver, signal strengths of nearby WLAN access points, or signal strengths of nearby mobile radio transceives), and/or ambient conditions, which ambient conditions comprise an air temperature, pressure and/or humidity.

Additionally, depending on the embodiment, the wearable device 2 comprises a processing unit separate from the processor 11. The processing unit is, for example, implemented as a microprocessor running program code (for example, embedded program code. As such, the processing unit may be configured to perform one or more steps and/or functions as described herein. The processing unit is connected to the sensor system and other electronic components of the wearable device 2, including the battery, user interface, communication module, etc.

Additionally, depending on the embodiment, the wearable device 2 comprises a memory connected to the processing unit configured to record a sensor output, comprising an output of the one or more sensors of the sensor system 3. Depending on the embodiment, the memory is integrated into the processing unit and/or the sensor system 3. In particular, the memory is configured to record the core body temperature values and the skin temperature measurement values. The memory may be further configured to record one or more additional measurement values as measured by the sensor system 3, in particular from those optional additional sensors of the sensor system described with reference to FIG. 10 below.

Depending on the embodiment, the sensor output from one or more sensors of the sensor system 3 is pre-processed (e.g., adjusted, corrected, filtered, statistically analyzed, summarized, compressed, and/or combined with an output of one or more other sensor outputs) in the wearable device 2.

Depending on the embodiment, the sensor output from a sensor of the sensor system 3 may be used to directly determine one or more physiological signals of the mammal. For example, a skin temperature sensor may directly measure the skin temperature, or an ECG sensor may be used to determine both the heart rate and the heart rate variability.

The sensor output from the sensor of the sensor system 3 may also be combined with one or more other sensor outputs from other sensors of the sensor system 3 to determine a physiological signal of the mammal. For example, the sensor output from a plurality of skin temperature sensors may be combined to determine an average skin temperature. In another example, the core body temperature measurement may be determined using a sensor output from one or more sensors (i.e. as a function of a sensor output from one or more sensors), in particular sensor output from a heat flux sensor 31 and a sensor output from a skin temperature sensor 32. The core body temperature measurement value may also be adjusted using a calibration file.

The skin temperature sensor may directly provide the sensor output from the skin temperature sensor 32. However, the skin temperature measurement value may also be corrected based on the position where the skin temperature sensor 32 is worn. The skin temperature measurement value may also be adjusted using a calibration file.

The sensor output from the sensor system 3 may be pre-processed, for example using LOWESS (Locally Weighted Scatterplot Smoothing), sometimes called LOESS (locally weighted smoothing) to remove outliers.

In another example, a plurality of sensor output values and/or physiological signal values are recorded over a pre-defined time interval and averaged to generate a single sensor output value and/or physiological signal value, which is then recorded in the memory. Alternatively, or additionally, a representative sensor output value and/or physiological signal value is selected as the sensor output value and/or physiological signal value, respectively. Preferably, sensor output values and/or physiological signal values are averaged across a period of ten seconds and recorded as a single sensor output value and/or a single physiological signal value, respectively. The skilled person is aware that other periods of time in the same general orders of magnitude (i.e. from approximately 1 second to approximately 100 seconds) are, depending on the embodiment, more appropriate than the example value of ten seconds given above. By not recording every single sensor output value and/or physiological signal value, the amount of data stored in the memory of the wearable device 2 is reduced. Further, the amount of data transmitted to the processor 11 is also reduced. Some sensor output values may be averaged across different time periods, for example while heart rate may be measured (or averaged) every 10 seconds while core body temperature and skin body temperature are measured (or averaged) every 30 seconds.

In an embodiment, the sensor system 3 is configured to record the output of a particular sensor only and/or record a particular physiological signal only when a particular set of circumstances apply. These circumstances include, for example, when the individual is active (which may be determined by an accelerometer and/or a heart rate), when the core body temperature and/or skin temperature is elevated (i.e. when the core body temperature exceeds a pre-defined threshold and/or the skin temperature exceeds a pre-defined threshold), when individual is exposed to heat strain and/or heat stress, and/or when a signal is received in the wearable device 2 to begin recording. In particular, the recording of the core body temperature and the skin temperature may depend on the heart rate being elevated, in particular higher than a pre-defined heart rate threshold, thereby indicating that the mammal is active.

Depending on the embodiment, the functions and/or steps described above related to adjustment, correction, pre-processing, filtering, statistically analyzing, and/or combining sensor outputs are executed in the wearable device 2 itself (in particular in the processing unit of the wearable device 2) or in the processor 11 of the electronic system 1.

Depending on the embodiment, the sensor system 3 of the wearable device 2 further comprises sensors configured to measure a heart rate, a heart rate variability, a skin perfusion, a breathing rate, respiratory rate, and/or aspects of electro dermal activity.

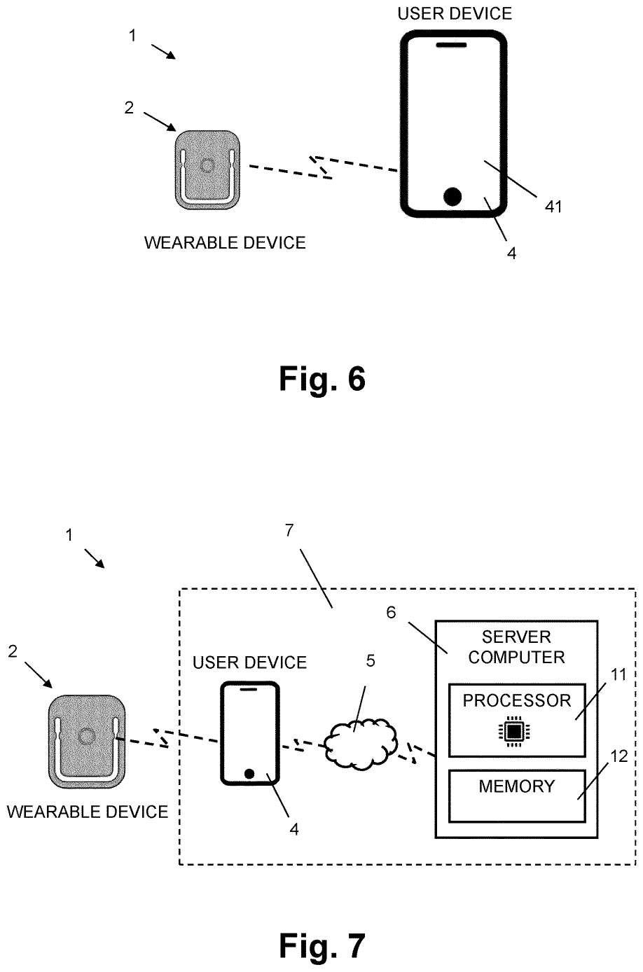

As shown in FIG. 4, the electronic system 1 comprises the wearable device 2, the processor 11, and the memory 12. The electronic system 1 may be implemented in a single device, or may be implemented in a plurality of devices communicatively coupled to each other, for example using the data communication system as described herein. For example, the electronic system 1 is implemented as a wearable device 2 connected to a user device 4 (as described in FIG. 6 in more detail).

As shown in FIG. 5, the electronic system 1 comprises a wearable device 2, the wearable device 2 including the sensor system 3, the processor 11, and the memory 12. The wearable device 2 may be implemented in a single device, or may be implemented in a plurality of devices communicatively coupled to each other, for example using the data communication system as described herein. For example, the electronic system 1 is implemented as a wrist worn device, for example an electronic bracelet or an electronic watch. Popular examples of such wrist worn devices include fitness trackers and smart watches.

As shown in FIG. 6, the electronic system 1 may be distributed between a wearable device 2 and a user device 4. One or more of the functions and/or steps described herein may be performed, in whole or in part, on the wearable device 2 and other functions and/or steps described herein may be performed, in whole or in part, in the user device 4. In particular, the steps and/or functions described herein as being performed on the processor 11 may be performed either in the wearable device 2 (the processor 11 being implemented in the wearable device 2) or in the user device 4 (the processor 11 being implemented in the user device 4). Additionally, in the case that the processor 11 is implemented in the user device 4, the wearable device 2 may have a separate processing unit as described herein, with the functions and/or steps described herein being performed either in the processing unit of the wearable device 2, the processor of the user device 4, or in conjunction between the two devices 2, 4.

Depending on the embodiment, the user device 4 is implemented as a mobile device, for example a mobile radio phone (e.g., a smart phone running an iOS or Android operating system), a tablet computer, a laptop computer, a smart watch, a fitness watch, a sports computer (e.g., an electronic sports watch, a cycling computer, or a rowing computer). The user device 4 may include a display 41, for example implemented as a touch screen. The user device 4 is configured to be connected to the wearable device 2 either using a wired or wireless data communication system.

As shown in FIG. 6, the electronic system 1 includes a wearable device 2, a user device 4, and a server computer 6. The wearable device 2 is connected to the user device 4 using the data communication system, in particular a using a short range wireless communications protocol such as Bluetooth LE (low energy) or ANT+. The user device 4 is connected to the server computer 6 using a data communication system, in particular via an intermediary network 5 which may include a mobile radio network and/or the Internet. The server computer 6 may be located remotely from the wearable device 2 and the user device 4, for example in a data processing facility such as a cloud computing center.

As depicted in FIG. 7, the processor 11 and the memory 12 may be implemented in the server computer 6. However, at least some of the steps and/or functions performed by the processor 11 may be performed, in whole or in part, in the user device 4 and/or in conjunction with the user device 4. For example, the user device 4 may perform pre-processing of the sensor data or physiological signals. The user device 4 may merely forward data received from the wearable device 2 to the server computer 4 (forwarding the data may include buffering, e.g., temporarily storing, the data). For example, the server computer 6 may perform data processing and transmit messages to the user device 4 based on the processed data. This is depicted in by the dashed box which indicates a distributed processing environment 7.

As depicted in FIG. 8, the electronic system 1 may comprise a wearable device 2 implemented as a smart device, in particular a smart watch comprising a user interface 23 (for example including a touch screen). The wearable device 2 is connected to the server computer 1 via an intermediary network 5 which may include a mobile radio network the Internet. To that end, the wearable device 2 includes a mobile radio transceiver, for example configured to communicate using one or more digital cellular technologies (e.g., GSM, GPRS, CDMA2000, EDGE, and/or UTMS). The steps and/or functions described herein may be performed by the wearable device 2, the server computer 6, and/or a combination of both.

FIGS. 9 and 10 show block diagrams illustrating schematically a sensor system 3 of the wearable device 2. As described, the sensor system 3 may be wholly integrated into the wearable device 2 (integrated in the sense of structural integration, e.g., that they share a common housing with the wearable device 2) or may be partially integrated, such that at least some sensors are integrated into the wearable device 2 while some other sensors are separately arranged and connected to the wearable device 2 using a data communication system as described herein.

The sensor system 3 is configured to measure physiological signals of the mammal, in particular the human. A sensor output of a particular sensor of the sensor system 3 may directly provide a measurement of one or more physiological signals, such as a skin temperature sensor directly providing the physiological signal of the skin temperature of the human. In another example, a sensor output of a particular sensor may directly provide a measurement of multiple physiological signals, for example a electrocardiogram (ECG) may provide a heart rate and a heart rate variability, or a photo plethysmograph (PPG) may provide a heart rate, a heart rate variability, a skin perfusion, and/or a breathing rate.

In yet another example, a sensor output of a particular sensor may be combined with another sensor output of another sensor to determine a physiological signal, such as the core body temperature being determined using a sensor output of a heat flux sensor and a sensor output of a skin temperature sensor. A mean body temperature may be determined using a combination of the core body temperature and the skin temperature, for example.

As depicted in FIG. 9, the sensor system 3 includes a heat flux sensor 31 and a skin temperature sensor 32.

The heat flux sensor 31 is preferably implemented using a Seebeck element, in particular comprising a series of p- and n-doped semiconductors. The heat flux sensor 31 and the skin temperature sensor 32 are preferably implemented using the sensor unit disclosed in WO2018114653A1, which publication is included herein by reference in its entirety.

The heat flux sensor 31 may alternatively be implemented using a plurality of temperature sensors. For example, the plurality of temperature sensors includes a first temperature sensor arranged such that it is in contact with the body, in particular in contact with the skin, when the wearable device 2 is worn, and a second temperature sensor arranged with a thermally conductive layer of a pre-determined thermal conductivity between the first and second temperature sensor, the second temperature sensor preferably being exposed to the environment (i.e. by being arranged on or in thermal contact with a surface of the wearable device 2 opposite the first temperature sensor).

The sensor system 3 may further include a photo plethysmograph (PPG) sensor. The PPG sensor may be integrated in the same device as the other sensors, for example in the wearable device 2, or be arranged separately from the other sensors. In particular, the PPG sensor may be implemented in a wrist worn device, such as a fitness tracker or smart watch. The PPG sensor may alternatively be arranged in an electronic ring worn on a finger. The PPG sensor is configured to communicate, using the data communication system as described herein, depending on the embodiment, with the wearable device 2, with the user device 4, and/or with the processor 11.

The sensor system 3 may further include an ECG sensor. The ECG sensor may be integrated in the same device as the other sensors of the sensor system 3, for example in the wearable device 2, or be arranged separately from the other sensors. In particular, the ECG sensor may be implemented in a second wearable device worn in contact with the torso, in particular the chest, of the human. The second wearable device may be attached to the human by way of a strap or belt. The (first) wearable device 2 may be attached to the human on the same strap of belt. The ECG sensor is configured to communicate, using the data communication system as described herein, depending on the embodiment, with the wearable device 2, with the user device 4, and/or with the processor 11.

Depending on the embodiment, the sensor system 3 may be configured to determine additional physiological signals using one or more sensors, including a galvanic skin response, a sweat rate, a breathing rate, an oxygen saturation, a blood oxygen saturation, a blood pressure, a glucose concentration, or a lactate concentration.

The sensor system 3 may further be configured to measure other values, including environmental conditions, such as an ambient air temperature using an ambient temperature sensor 33, and an ambient humidity, or values related to a location or movement of the human. The location may be determined using a GNSS receiver (e.g. a GPS receiver). The movement may be determined by means of an inertial measurement unit (IMU) comprising, for example, a three axis accelerometer, a gyroscope, and/or a magnetic field sensor.

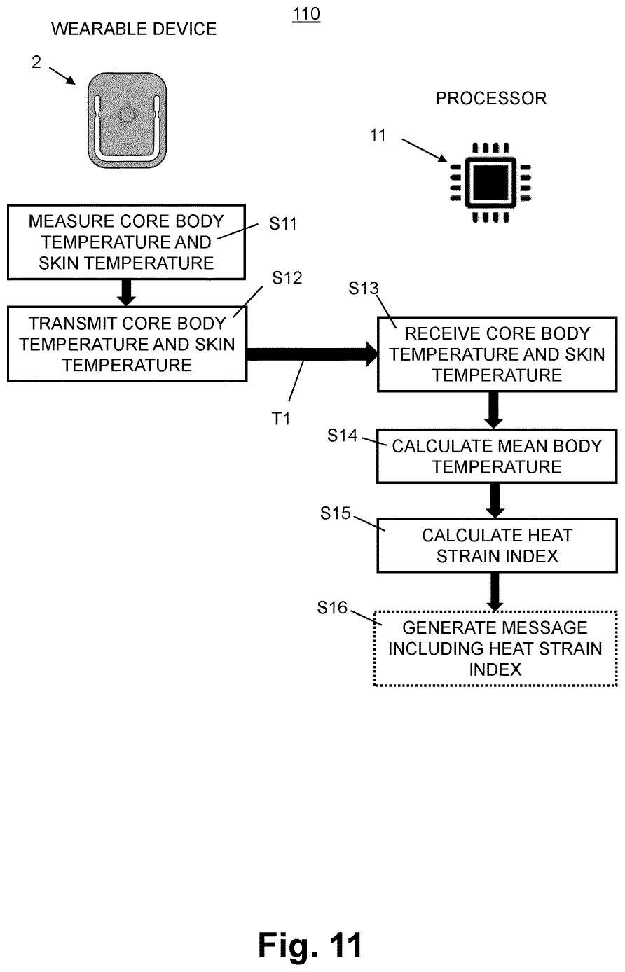

FIGS. 11 to 15 show flow diagrams illustrating methods 110, 120, 130, 140, 150, each comprising a number of steps performed by the electronic system 1, in particular the wearable device 2 and/or the processor 11. These methods 110, 120, 130, 140, 150 may be performed during and/or after a period of elevated heat, i.e. a period of time during which the mammal experiences a physiological strain due to heat. The physiological strain may be due to externally induced environmental heat and/or may be due to elevated bodily activity. The period of elevated heat may be a training session, in the case of an athlete, or a work shift in the case of a worker. The methods 110, 120, 130, 140, 150 or particular steps may be performed once, in an intermittent fashion, or in a continuous fashion. The methods 110, 120, 130, 140, 150 or particular steps thereof may take place on demand, i.e. when an appropriate signal is received from a user or from a device which triggers the method or steps, or the methods or particular steps thereof may be performed automatically, for example at pre-determined times or after pre-determined periods.

FIG. 11 shows a flow diagram illustrating a method 110 which includes a number of steps S11-S15 for calculating a heat strain index.

In step S11, the wearable device 2, in particular the sensor system 3 of the wearable device 2, measures a core body temperature CBT and a skin temperature ST of the mammal, in particular the human.

The core body temperature CBT may be determined using a heat flux HF and a skin temperature ST. Preferably, the heat flux HF is measured using a heat flux sensor in contact with the mammal, in particular in contact with the skin of the human. Preferably, the heat flux sensor is arranged at the same location on the body as the skin temperature sensor, e.g., inside the same wearable device. Specifically, the core body temperature CBT is determined as a function of the heat flux HF and the skin temperature ST, in particular using known algorithms or models, for example using a resistive model of heat flow from a core of the body to the environment via the skin. For example, using the following function:

$$C B T = S T + R B \times H F ,$$

where RB is the thermal resistance of the human body at the defined location on the human body.

In another example, the core body temperature CBT is determined from the skin temperature ST and the heat flux HF using a statistical algorithm based on the skin temperature ST and the heat flux HF that has been generated using machine learning.

The core body temperature CBT may also be determined using a temperature sensor in the form of a swallowable device with a wireless transponder which wirelessly transmits the core body temperature to the wearable device 2.

In step S12, the core body temperature CBT and the skin temperature ST are transmitted, in a transmission T1, from the wearable device 2 to the processor 11. The transmission T1 is a wired or wireless transmission, in particular using the data communication system as described herein. In an embodiment where the processor 11 and the wearable device 2 are integrated, i.e. part of the same device, steps S12 and S13 may be omitted.

The wearable device 2 may be configured to perform steps S11 and S12 continuously, for example at 1 second intervals. As described herein, the wearable device 2 may be configured to pre-process the core body temperature CBT and the skin temperature ST.

Depending on the embodiment, the wearable device 2 may be configured to transmit other sensor outputs or other physiological signals as described herein to the processor 11. In particular, the transmission T1 from the wearable device 2 to the processor 11 is a transmission from a communication module of the wearable device 2 to a communication module connected to the processor 11. For example, the communication module connected to the processor 1 may be a communication module of the user device 4 or the server computer 6. The transmission T1 may be direct or indirect, for example a direct transmission to the server computer 6 using a mobile radio network, or an indirect transmission via the user device 4.

In step S13, the processor 11 receives the core body temperature CBT and the skin temperature ST from the wearable device 2, in particular using the data communication system.

In an embodiment, the processor 11 applies a correction to the skin temperature ST received from the wearable device 2. Alternatively, the wearable device 2 is configured to apply the correction, and transmit the (corrected) skin temperature ST to the processor 11. The skin temperature ST is corrected using a correction term, the correction term being associated with a defined position on which the wearable device 2, in particular the skin temperature sensor 32, is arranged on the body 8. Different positions may have different correction terms.

In an embodiment, the correction term is an offset value of 0.3° C.-1° C. The precise value of the offset value depends on the defined position. For example, where the defined position is a chest area of the torso, more particularly an apical location (located on the left side of the chest between the left armpit and the left hip), the offset value is 0.4° C.-0.8° C., preferably 0.5° C.

In an embodiment wherein the defined position is the arm, preferably the upper arm or the wrist, the defined offset value is 0.6° C.-1.0° C., preferably 0.7° C.

The corrected skin temperature cST is calculated, for example, using the following function:

$$c S T = ST - offset ,$$

where ST is the skin temperature and offset is the offset value.

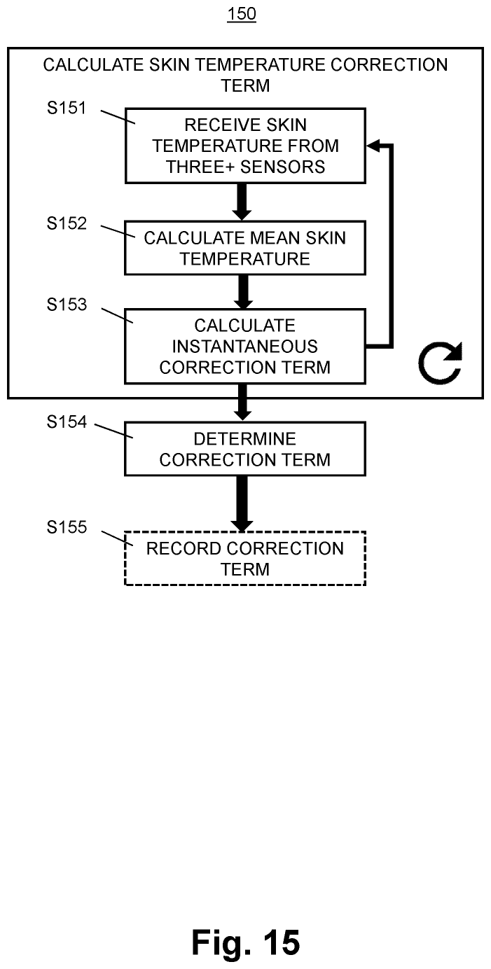

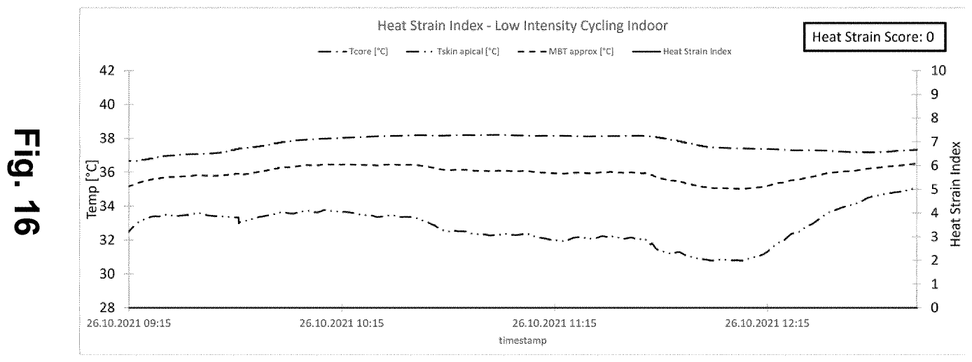

In an embodiment, the defined position is selected from one of the following positions: the torso, the upper arm, or the wrist, and the defined correction term is an individual correction term, determined for the particular human. A method for determining the individual correction term is discussed in more detail with reference to FIG. 15.

In step S14, the processor 11 calculates a mean body temperature MBT using the core body temperature CBT and the skin temperature ST. The mean body temperature MBT is a value representing a mean body temperature of the mammal which correlates well with a physiological strain due to heat.

In particular, the processor 11 calculates the mean body temperature MBT as a function of the core body temperature CBT and the skin temperature ST.

In particular, the processor 11 calculates the mean body temperature MBT as a linear combination of the core body temperature CBT and the skin temperature ST, preferably a weighted sum according to the following formula:

$$MBT = X \cdot CBT + Y \cdot ST ,$$

where MBT is the mean body temperature, CBT is the core body temperature, ST is the skin temperature (alternatively, the correct skin temperature can also be, and X and Y are coefficients of the core body temperature and the skin temperature, respectively. Preferably, the coefficients are defined such that the weighted sum is a normalized weighted sum. The coefficient X has a value of 0.6-0.7, preferably 0.64. The coefficient Y has a value of 0.3-0.4, preferably 0.36.

In step S15, the processor 11 calculates a heat strain index HSI. The heat strain index HSI is a measure of the physiological strain due to heat. The heat strain index HSI increases as a function of the mean body temperature MBT. For example, the HSI can be defined to be proportional to the MBT. The HSI can alternatively, or additionally, be defined as a sum of a number of terms, for example as a polynomial function of the mean body temperature.

In an embodiment, the heat strain index HSI is calculated such that, when there is no physiological strain due to heat, the HSI has a value of zero. The heat strain index HSI may be calculated as a function of the difference between a current mean body temperature MBT and a resting mean body temperature rMBT.

The resting mean body temperature rMBT may also be used to calculate the lower temperature threshold LTT. For example, the lower temperature threshold LTT may be defined as being 0.5° C.-1.0° C., preferably 0.7° C., higher than the resting mean body temperature rMBT. The lower temperature threshold LTT can alternatively be a general pre-determined value, in particular the lower temperature threshold LTT can be a specific pre-determined value which depends on a group which the human belongs to, for example an occupation, a climate zone, an age, or a sport, or a value individualized for the human.

In the case of mammals other than humans, a mammal specific lower temperature threshold LTT may be used.

The lower temperature threshold LTT individualized for the human may be determined by recording the mean body temperature MBT during a period where there is little to no physiological strain due to heat, for example during rest and/or sleep, and defining the lower temperature threshold LTT using the lowest value of the mean body temperature MBT recorded during such period, or using a low percentile value of the mean body temperature MBT recorded during such period (the low percentile value being between the 1^{st} and the 10^{th} percentile of the mean body temperature MBT, for example). As mentioned above, the lower temperature threshold LTT may be defined as being 0.5° C.-1.0° C., preferably 0.7° C., higher than the resting mean body temperature rMBT. The lower temperature threshold LTT a value that typically lies in the range 36° C.-37° C. A pre-defined value for the lower temperature threshold LTT which works well for humans is 36.7° C.

In an embodiment, the heat strain index HSI is calculated using the mean body temperature MBT, the lower temperature threshold LTT, and an upper temperature threshold UTT. The upper temperature threshold UTT is a temperature value which represents a mean body temperature above which it is likely that the mammal, in particular the human, will begin to experience serious symptoms due to the heat strain, such as heat syncope, heat cramps, and heat exhaustion. The upper temperature threshold UTT is a value that typically lies in the range 38° C.-40° C. A value for the upper temperature threshold UTT which works well for humans is 38.9° C.

For example, the heat strain index HSI is calculated as a function of the mean body temperature MBT, the lower temperature threshold LTT, and the upper temperature threshold UTT. In particular, the heat strain index HSI is calculated such that it is proportional to a difference between the mean body temperature MBT and the lower temperature threshold LTT, and inversely proportional to a difference between the upper temperature threshold UTT and the lower temperature threshold LTT, for example according to the following function:

$$HSI ∝ \frac{M T B - L T T}{U T T - L T T}$$

The proportionality factor may be chosen as desired. For example, the scaling factor may be 10 or 100, such that the heat strain index HSI is a value that typically lies between 0 and 10, or between 0 and 100, respectively.

In an embodiment, the heat strain index HSI further includes a heart rate component HRC which depends on heart rate HR, in particular on a current heart rate HR of the individual. For example, the heart rate component HRC is calculated using a current heart rate HR, a resting heart rate rHR and a max heart rate mHR, for example according to the following formula:

$$HRC ∝ \frac{HR - rHR}{mHR - rHR}$$

For example, the heart rate component is included in the calculation of the heat strain index HSI as a summed factor and/or as a coefficient. For example,

$$HSI ∝ \frac{MTB - LTT}{UTT - LTT} + HRC , or$$

$$HSI ∝ \frac{MTB - LTT}{UTT - LTT} \times HRC .$$

In an embodiment, the heat strain index HSI is calculated further using other physiological signals, in particular heart rate, heart rate variability, galvanic skin response, sweat rate, breathing rate, oxygen saturation, heat flux, movement data, glucose concentration, or lactate concentration. These further physiological signals may be implemented in the form of one or more coefficients which modify the calculated heat strain index HSI, and/or one or more constant values which are added to or subtracted from the heat strain index.

Additionally or alternatively, these physiological signals may also be used to modify the lower temperature threshold LTT and/or the upper temperature threshold UTT.

In an embodiment, the heat strain index HSI is calculated further using individual factors such as age, sex, physiological state, and/or health. These factors may be implemented in the form of one or more coefficients which modify the calculated heat strain index HSI, and/or one or more constant values which are added to or subtracted from the heat strain index.

Additionally or alternatively, these factors may also be used to modify the lower temperature threshold LTT and/or the upper temperature threshold UTT.

In an optional step S16, the processor 11 generates a message depending on the heat strain index HSI. The message may be recorded in the memory 12. The message may also be transmitted back to the wearable device 2, to the user device 2, and/or to the server computer 6. The message may be transmitted only if the heat strain index HSI exceeds a pre-defined heat strain index threshold.

The message may include the heat strain index HSI, for example as a numerical value. Alternatively or additionally, the message may include a representation of the HSI, for example a text, color, or audio signal which depends on the heat strain index HSI. An example of possible representations is shown in the table below, for a heat strain index HSI with a scaling factor of 10.

| HSI | Text | Color |
|---|---|---|
| 0-3 | Low Heat Strain | Green |
| 3-7 | Middle Heat Strain | Yellow |
| 7-10 | Severe Heat Strain | Red |

Depending on the precise implementation, the granularity may be higher or lower, as required.

The message may further include an alarm, warning, and/or alert, depending on the value of the heat strain index HSI. The message may be configured such that it is displayed as an important notification on the user device 4, for example. The message may be configured to be displayed prominently, and may include the use of signal colors, i.e. conspicuous colors such as orange or red that have a signal effect and which are interpreted by a substantial proportion of humans as a warning signal.

In an embodiment, the electronic system 1 is configured to display the message on a display, for example a display 23 of the wearable device 2, and/or on a display 41 of the user device 4.

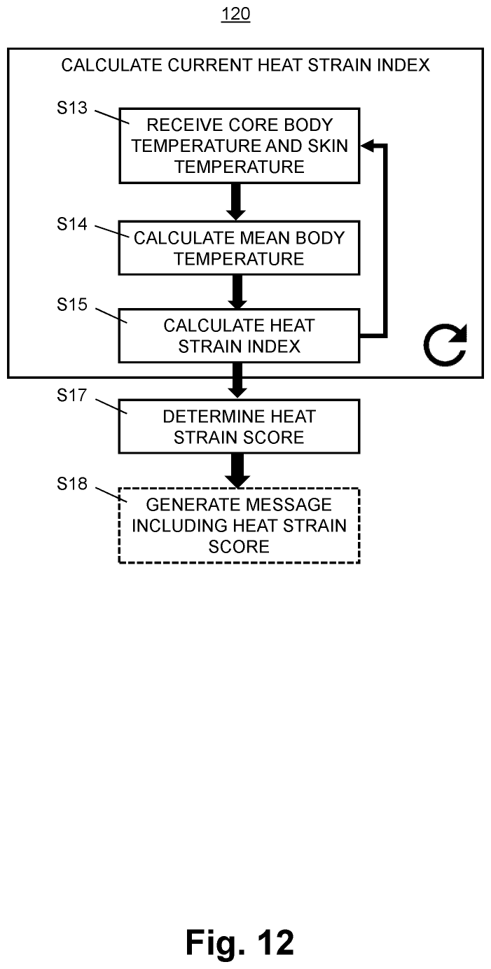

FIG. 12 shows a flow diagram illustrating a method 120 including a number of steps S13-S15 which are described above with reference to FIG. 11, as well as further steps S17, S18. As depicted in the flow diagram, the steps S13-S15 are performed continuously, for example once every second or once every ten seconds.

In step S17, the heat strain index HSI calculated in step S15 is used to determine, in the processor 11, a heat strain score HSS. The heat strain score HSS is an indicator of a cumulative physiological strain due to heat and therefore can be a useful indicator for purposes of monitoring a total heat exposure to ensure that the physiological strain is not too great, or for monitoring acclimatization, to ensure that the total dose of heat exposure is high enough to elicit a physiological adaptation, but not high enough to be detrimental to future performance, recovery, or acclimatization.

The heat strain score HSS may be calculated “live”, that is, calculated during a period of elevated heat based on a current value of the heat strain index HSI and past values of the heat strain index HSI during the current period of elevated heat. The heat strain score HSS may also be calculated retrospectively, that is, calculated subsequent to the period of elevated heat based on a plurality of values of the heat strain index HSI calculated from (and/or recorded during) the mean body temperature MBT during the period of elevated heat.

The heat strain score HSS is calculated as a function of the average value of the heat strain index HSI during the period (or part thereof) and a duration P of the period (or part thereof) of elevated heat. The duration P may be the entire period of elevated heat, a duration P up to the current present moment, or a subset of the entire period. The duration P may also be selected as an entire day, for example. For example, the heat strain score HSS is calculated using the following formula:

$$H S S = aHSI \times P ,$$

where aHSI is an average of the heat strain index HSI. The average heat strain index aHSI may be calculated as a mean of a plurality of values of the heat strain index HSI recorded at regular time intervals (for example, every 1-60 seconds, preferably every 1-30 seconds) according to the following formula:

$$Average HSI = \frac{{\sum }_{n} {HSI}_{n}}{n} ,$$

where n is the number of values of the heat strain index HSI.

Alternatively, the heat strain score HSS is calculated as a function of a normalized heat strain index nHSI during the period (or part thereof) and a duration P of the period (or part thereof) of elevated heat according to the following formula:

$$HSS = nHSI \times P .$$

The normalized heat strain index nHSI is calculated by taking an i-th power of multiple values of the heat strain index HSI, preferably regularly spaced in time (for example, every 1-60 seconds, preferably every 1-30 seconds). The index i may be a natural or a real number, preferably between 1 and 5, most preferably 4. A mean is then calculated of i-th powers, and the i-th power then taken of the mean. For example, the normalized heat strain index nHSI is calculated using the following formula:

$$nHSI = \sqrt[i]{\frac{{\sum }_{n} {HSI}_{n}^{i}}{n}} ,$$

where n is the number of values of the heat strain index HSI. By normalizing the heat strain index HSI in such a manner and calculating the heat strain score HSS using the normalized heat strain index nHSI, higher values of the heat strain index HSI contribute proportionally more to the heat strain score HSS than lower values. This reflects the observation that the physiological strain due to heat does not increase linearly with increased mean body temperature MBT but rises exponentially as the mean body temperature MBT increases.

The value of the heat strain score HSS obtained according to step S17 may be used as a guide for comparing two periods of elevated heat with different profiles of the mean body temperature MBT or different durations P. The results will, however, depend on whether the average heat strain index aHSI is used or the normalized heat strain index nHSI. For the purposes of the following examples, a value of the heat strain index HSI every ten seconds is used for determining the heat strain score HSS, thereby resulting in 600 values of the heat strain index HSI per hour.

A first exemplary training session has a duration P of two hours, both hours at a heat strain index of 6. Therefore, the average heat strain index aHSI for this training session would be 6. This results in a heat strain score HSS of aHSI×100×P=1200, with the factor of 100 being selected such that an average heat strain index aHSI of 10 for an hour results in a heat strain score HSS of 1000.

A second exemplary training session with a duration P of two hours and a heat strain index HSI of 4 during the first hour and 8 during the second hour would also result in the average heat strain index aHSI of 6, and therefore the heat strain score HSS calculated using the average heat strain index aHSI would logically result in the same heat strain score HSS of 1200 as the first training session. However, the total physiological strain of both sessions is not the same. In particular, the second session has a higher physiological strain due to the higher value of the heat strain index HSI of the second hour, which results in a disproportionately higher physiological strain.

Using the normalized heat strain index nHSI, however, a more accurate measure of the total physiological strain may be found, which corresponds more closely to the physiological strain perceived by the human. Using the normalized heat strain index nHSI and a value of i=4, the first training session would result in a heat strain score HSS of 1200, thereby matching the result of the calculation based on using the average heat strain index aHSI. However, the second training session would result in a heat strain score of 1366, thereby reflecting the greater physiological toll of the second training session.

The heat strain score HSS may be subject to a scaling factor. The scaling factor can depend on the time resolution of the values of the heat strain index HSI used, i.e. the time period between subsequent values of the heat strain index HSI. For example, the scaling factor can be selected such that the heat strain score HSS generated during a training session of up to one hour is not likely to exceed 1000.

In the optional step S18, the processor 11 generates a message including the heat strain score HSS. The message may be recorded in the memory 12. The message may be transmitted to the wearable device 2, for example for displaying on the display 23 of the wearable device 2.

The message may also be transmitted back to the wearable device 2, to the user device 4, and/or to the server computer 6. The message may be transmitted only if the heat strain score HSS exceeds a pre-defined heat strain score threshold.

The message may include the heat strain score HSS, for example as a numerical value. Alternatively or additionally, the message may include a representation of the heat strain score HSS, for example a text, color, or audio signal which depends on the heat strain score HSS. An example of possible representations is shown in the table below, for a heat strain score HSS scaled such that a training session of up to one hour is not likely to exceed 1000 (i.e., a score of 1000 would require that the heat strain index HSI is 10 for an entire hour).

| HSS | Text | Color |
|---|---|---|
| 0-300 | Low Heat Strain Score | Green |
| 301-700 | Moderate Heat Strain Score | Yellow |
| 701-1000 | Severe Heat Strain Score | Red |
| 1000+ | Seek Medical Attention | Violet |

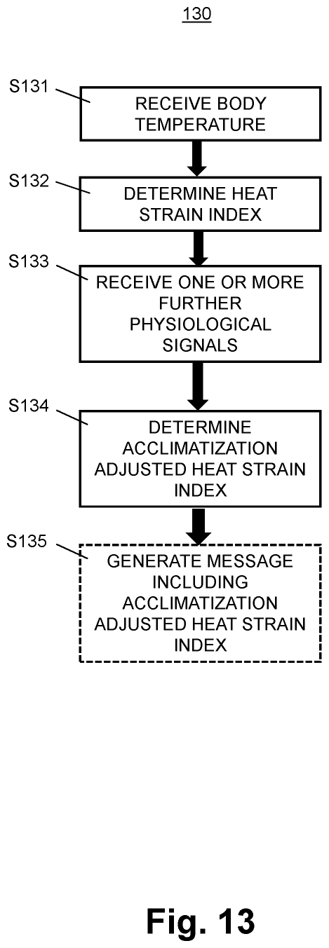

FIG. 13 shows a flow diagram illustrating a method 130, including number of steps S131-S135 for determining an acclimatization adjusted heat strain. The method 130 may be performed by the electronic system 1. The acclimatization adjusted heat strain takes into account further physiological signals than just the mean body temperature, such that it is possible to account for the degree of acclimatization for an individual more accurately and robustly than just relying on the core body temperature.

In step S131, a body temperature is received. Preferably, the body temperature is a mean body temperature MBT, in particular the mean body temperature MBT calculated as described herein, for example using a core body temperature CBT and a skin temperature ST.

In step S132, a heat strain index HSI is determined using the body temperature. Preferably, the heat strain index HSI is determined according to the method 110 as described herein.

In step S133, one or more further physiological signals of the mammal, in particular of the human, are received. The physiological signals are preferably those physiological signals which vary due to the response of the body to heat, in particular those which further depend on a degree of acclimatization. The physiological signals include, for example, heart rate, heart rate variability, and/or respiration rate (i.e. breathing rate). The physiological signals may further include a galvanic skin response, hydration status, body weight, a sweat rate, a salt and/or electrolyte concentration in sweat, a perfusion, and/or other aspects of electro dermal activity.

In step S134, an acclimatization adjusted heat strain index aaHSI is determined using (e.g., as a function of) the heat strain index HSI and one or more of the further physiological signals.

For example, the acclimatization adjusted heat strain index aaHSI is determined using the heat strain index HSI and the heart rate HR. For example according to the following formula:

$$aaHSI = HSI + {k}_{HR} \frac{HR - {HR}_{R}}{{HR}_{ma x} - {HR}_{R}} ,$$

where HR is the heart rate, HR_{R} is the resting heart rate, HRₘₐₓ is the max heart rate, and k_{HR} is a heart rate coefficient which may be a value between −1 and +1.

In general, the acclimatization adjusted heat strain index aaHSI is determined using the heat strain index HSI and one or more coefficients and/or offset factors, which coefficients and/or offset factors are calculated using the further physiological signals. In particular, the following formula may be used:

$$aaHSI = ( {PC}_{A 1} \times {PC}_{A 2} \times … \times {PC}_{An} ) HSI + ( {PC}_{B 1} + {PC}_{B 2} + ⋯ + {PC}_{B n} ) ,$$

where aaHSI is the acclimatization adjusted heat strain index, HSI is the heat strain index, PC_{A1} is a first physiological coefficient associated with a first physiological signal, PC_{A2} is a second physiological coefficient associated with a second physiological signal, and PC_{An} is an n-th physiological coefficient associated with an n-th physiological signal, and PC_{B1} is a first offset associated with a first physiological signal, PC_{B2} is a second offset associated with a second physiological signal, and PC_{Bn} is an n-th offset associated with an n-th physiological signal.

An example of how the physiological coefficient may be determined for a given physiological signal is given according to the following formula:

$$P {C}_{A 1} ∝ \frac{C P S V - L S T}{U S S - L S S} ,$$

where CPSV is a current physiological signal value, LST is a lower signal threshold and USS is an upper signal threshold. Similarly, an example of how a physiological offset may be determined for a given physiological signal is given according to the following formula:

$$P {C}_{B 1} ∝ \frac{C P S V - L S T}{U S S - L S S} .$$

The hydration status may be determined by measuring the sweat, in particular the sweat rate, a salt and/or electrolyte concentration in sweat, and/or a fluid loss. In particular, the hydration status may be determined using the sweat rate integrated over time. Further, the hydration status may be determined using a current body weight, in particular in an example where the body weight may be directly measured (e.g. an exercise bicycle, rowing machine, or treadmill which is configured to measure the body weight of the individual).

In an optional step S135, a message is generated including the acclimatization adjusted heat strain index aaHSI.

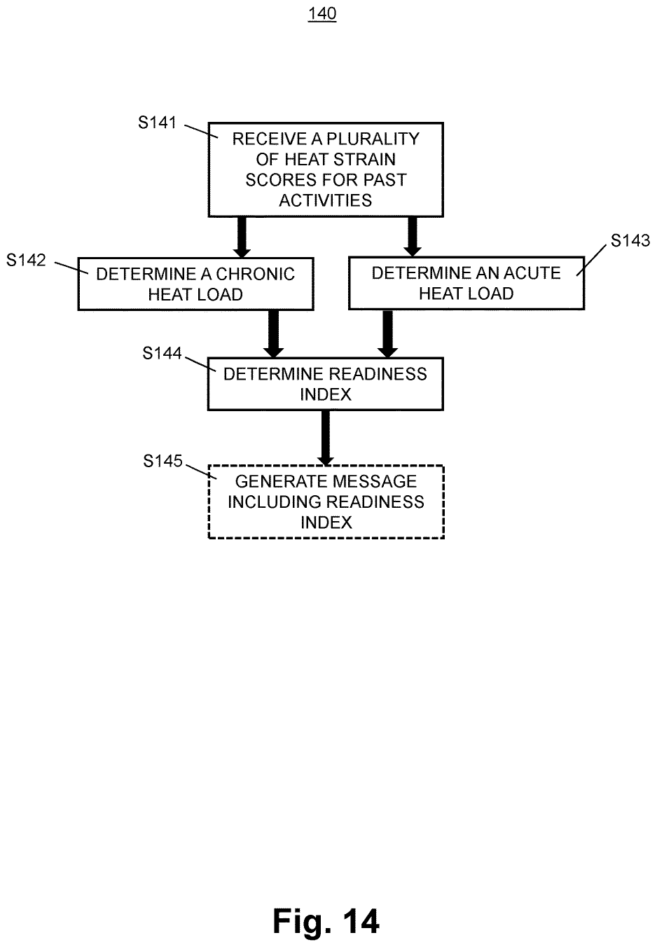

FIG. 14 shows a flow diagram illustrating a method 140 including a series of steps S141 to S145 for determining a readiness index for exposure to elevated heat. The method 140 may be performed by the electronic system 1 described herein.

In step S141, a plurality of heat strain scores HSS for past periods of elevated heat are received, for example past activities or training sessions. For example, the heat strain scores HSS are associated with past active training sessions aimed at increasing heat acclimatization, or activities with heat exposure, such as sauna, or working in hot environments.

In step S142 a chronic heat load CHL is determined. The chronic heat load CHL is a measure of the long term physiological toll which the body has experienced due to a prolonged history of past heat exposure(s). The chronic heat load CHL may be determined for a current time-point t, taking into account past heat exposure(s). The chronic heat load CHL may also be determined for a time-point t in the past, taking into account heat exposure(s) in the heat exposures further in the past with respect to the time-point t in the past.

In particular, the chronic heat load CHL is determined using a sum of the heat strain score HSS for a plurality of past periods of elevated heat, for example the past 5-40 days. Preferably, the sum is weighted such that periods of elevated heat further in the past are weighted less than periods of elevated heat closer to the current time-point. In particular, the weight of each heat strain score HSS depends on a time interval (i.e., a number of days) between the current time-point t and the time of the past period of elevated heat.

For example, the weight of a particular heat strain score HSS decreases linearly with time, for example such that the contribution of a particular heat strain score HSS becomes zero between 5-40 days.

For example, the weight of a particular heat strain score HSS decreases according to an exponential decay, for example with a time constant of 5-30 days. An example of for calculating the chronic heat load CHL at a given time t is given by the following formula:

$$C H L ( t ) = {k}_{1} {\sum }_{s = 0}^{t - 1} {e}^{- \frac{t - s}{{τ}_{1}}} H S S ( s ) ,$$

where CHL(t) is the chronic heat load on a particular time (e.g., day) t, HSS(s) is the heat strain score at a particular time (e.g., day) s prior to t, τ₁ is a time constant of 5 to 30 (days), and k₁ is a scaling factor.

The time constant τ₁ is a defined time constant that may vary between individuals, between an individual over time, or between groups of individuals. The time constant may be calibrated to a particular individual. The scaling factor k₁ may also vary between individuals or between groups of individuals.

The chronic heat load CHL is a measure of how heat acclimatized an individual is and to what degree physiological adaptations will have taken place to allow the individual to perform better under elevated heat. A high chronic heat load CHL is indicative of a high degree of acclimatization to heat. The chronic heat load CHL, in particular as determined using the exponentially weighted sum of past heat strain scores HSS, reflects that individuals gradually lose the physiological adaptations which let them perform under heat.

In step S143 an acute heat load AHL is determined. The acute heat load AHL is a measure of the physiological toll which the body has experienced due to very recent heat exposure(s). The acute heat load AHL may be determined for a current time-point, taking into account recent heat exposure(s). The acute heat load AHL may also be determined for a time-point t in the past, taking into account heat exposure(s) in the recent past with respect to the time-point in the past.

In particular, the acute heat load AHL is determined using a sum of the heat strain score HSS for a plurality of past periods of elevated heat, for example the past 2-14 days. Preferably, the sum is weighted such that periods of elevated heat further in the past are weighted less than periods of elevated heat closer to the current time-point. In particular, the weight of each heat strain score HSS depends on a time interval (i.e., a number of days) between the current time-point and the time of the past period of elevated heat.

For example, the weight of a particular heat strain score HSS decreases linearly with time, for example such that the contribution of a particular heat strain score HSS becomes zero between 2-14 days.

For example, the weight of a particular heat strain score HSS decreases according to an exponential decay, for example with a time constant of 2-10 days. An example of for calculating the acute heat load AHL at a given time t is given by the following formula:

$$A H L ( t ) = {k}_{2} {\sum }_{s = 0}^{t - 1} {e}^{- \frac{t - s}{{τ}_{2}}} H S S ( s ) ,$$

where AHL (t) is the acute heat load on a particular time (e.g., day) t, HSS(s) is the heat strain score at a particular time (e.g., day) s prior to t, τ₂ is a time constant of 2 to 10 (days), and k₂ is a scaling factor.

The time constant τ₂ is a defined time constant that may vary between individuals, between an individual over time, or between groups of individuals. The time constant may be calibrated to a particular individual. The scaling factor k₂ may also vary between individuals or between groups of individuals.

The acute heat load AHL is a measure of the current physiological toll which the individual is experiencing due to exposure to elevated heat in the very recent past. A high value of the acute heat load AHL is known to hinder performance as the body is still recovering from heat exposure.

In a step S144, a heat readiness index HRI is calculated, for a particular time-point t, using the chronic heat load CHL and the acute heat load AHL. In particular, the heat readiness index HRI is calculated as a linear combination of the CHL and the AHL, for example using the following formula:

$$HRI ( t ) = {R}_{0} + C H L ( t ) - A H L ( t ) ,$$

where HRI (t) is the heat readiness index at a particular time-point t, R₀ is a base readiness index which may vary from individual to individual, CHL(t) is the chronic heat load at a time-point t, and AHL is the acute heat load at a time-point t.

The heat readiness index HRI is a measure of how ready an individual is for performance under heat exposure. A high heat readiness index HRI requires not only a high value for the chronic heat load CHL (indicative of a high degree of acclimatization), but also a low value for the acute heat load (AHL), which is indicative of a current physiological toll due to recent heat exposure.

For example, to achieve a high heat readiness index HRI, an athlete may perform training sessions with a high heat strain index HSI for two or three weeks, thereby leading to a high chronic heat load CHL. However, for a few days before an event, the athlete may not perform any training sessions with a high heat strain index HSI, thereby allowing the athlete's body to recover and resulting in a lower value of the acute heat load AHL at the day of the event.

In an embodiment, a message is generated. The message may include an indicator of the acute heat load AHL, the chronic heat load CHL, and/or the heat readiness index HRI.

FIG. 15 shows a flow diagram illustrating a method 150 for determining the correction term for a particular individual, the correction term being used to correct the skin temperature ST as described herein. The defined correction term relates in general to a heat transfer coefficient between the skin and the environment, which varies across the skin surface and whose variation is different from individual to individual. The correction term determined hereby takes into account differences between individuals and provides and may further take into account clothing worn over the wearable device 2, in particular over the skin temperature sensor 32 of the sensor system 3. The method allows for correction terms to be determined for different defined positions. In particular, the method is used to find the correction term for the apical position, i.e. the position on the left side of the chest indicated by reference numeral 81 in FIG. 1. The method 150 includes a number of steps S151-S155. The steps may partly or wholly be performed in the processor 11, partly or wholly in the wearable device 2, or in conjunction between the processor 11 and the wearable device 2. Additionally, the user device 4 and/or the server computer 6 may also perform part or all of one or more of the steps S151-S155.

In a preparatory step, three or more, preferably four, skin temperature sensors 32 are attached to the body 8 of the mammal, in particular the human. The skin temperature sensors 32 are, for example, attached at defined positions on the body 8 of the human, in particular using a selection of the defined positions 81, 82, 83, 84, 85 as depicted in FIG. 1. The three or more skin temperature sensors 32 preferably communicate wirelessly with the wearable device 2 and/or a user device 4. The measured skin temperatures may be transmitted, from the skin temperature sensors 32 directly, or via an intermediary device, to the processor 11.

In step S151, three or more skin temperature measurement values are received from the three or more skin temperature sensors 32. The three or more skin temperature measurement values will not all be identical, as the sensors are placed on different parts of the body 8 which have a different skin temperature.

In step S152, an average (e.g., a mean) skin temperature value is calculated using the three or more skin temperature measurement values. The average skin temperature aST may be calculated using the following formula:

$$a S T = \frac{S {T}_{81} + S {T}_{82} + S {T}_{83} + S {T}_{84}}{4} ,$$

wherein the subscript of the skin temperature ST indicates the position 81, 82, 83, 84 on the body 8.

In step S153, the instantaneous correction term is calculated using the current value of the average skin temperature aST. The instantaneous correction term iCT is calculated for example as a difference between the desired defined position (e.g., the apical position 81) and the average skin temperature aST using the following formula:

$$i C T = S {T}_{81} - a S T .$$

The instantaneous correction term iCT is recorded. As indicated in FIG. 15, steps S151-S154 occur repeatedly for a particular duration of time, for example for the duration of a first heat training session. The instantaneous correction term iCT is recorded for the duration and then the correction term CT is derived therefrom.

In step S154, the correction term CT is determined using the recorded values of the instantaneous correction term iCT. The correction term CT may be determined using one or more averages (e.g., mean, median, and/or mode) of the instantaneous correction term iCT.

In step S155, the correction term CT is recorded in the electronic system 1. For example, the correction term CT is recorded in the memory of the wearable device 2 and/or in the memory 12 connected to the processor 11.

The method as described above therefore enables the electronic system 1 to apply, in future periods of elevated heat, an individualized correction term CT to measurements of the skin temperature taken at the defined position, for example the apical position 81.

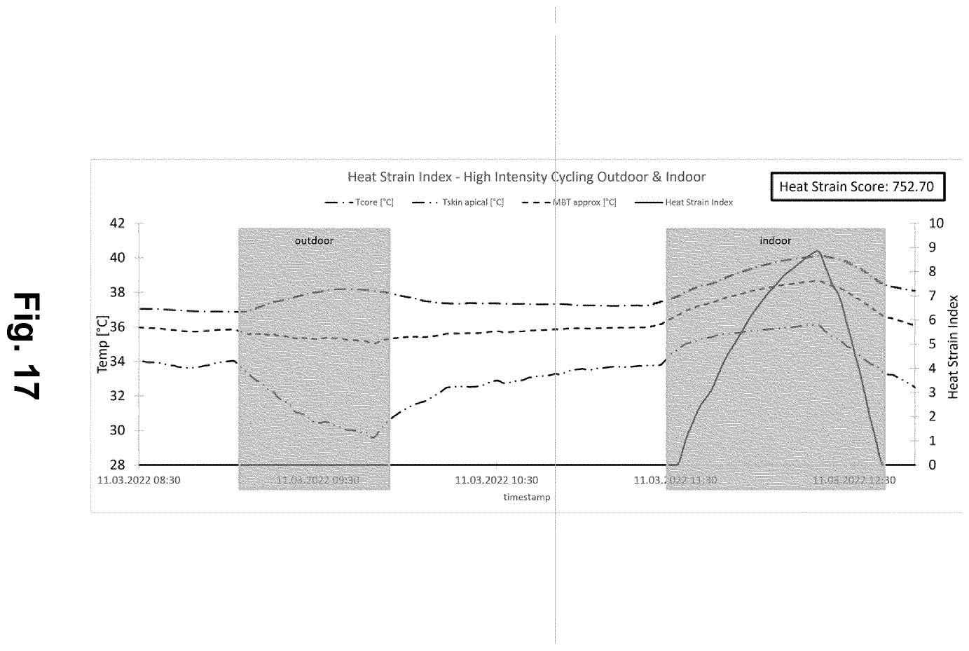

FIG. 16 shows a chart with time-series plots of sensor output values from a sensor system 3 of a wearable device 2 worn by an individual. The individual is cycling indoors at low intensity for a period of three hours. The core body temperature CBT measurement is shown to begin at just under 37° C., rise during the second hour to approximately 38° C. and begin to settle again in the third hour. The skin temperature ST begins under 33° C., initially rises, drops during the second and third hour before rising towards the end of the session to above 34° C. The mean body temperature MBT calculated using the core body temperature CBT and the skin temperature ST falls between the CBT and ST and rises initially from a value of approximately 35° C. towards 36° C., remains fairly steady during the first two hours, dropping in the third hour in response to the skin temperature ST dropping, before rising towards the end of the session back towards 36° C. The heat strain index HSI during the session remains 0 at all times as the mean body temperature MBT remains below the lower temperature threshold LTT. Therefore, the calculated heat strain score HSS for the session is also 0.

FIG. 17 shows a chart with time-series plots of sensor output values from a sensor system 3 of a wearable device 2 worn by an individual. The time-series plots cover four hours, including a first outdoor high intensity cycling session and a second indoor high intensity cycling session.

During the outdoor session, which has a duration of approximately one hour, the core body temperature CBT is seen to rise from an initial value of approximately 37° C. to over 38° C. before dipping slightly towards the end of the session. The skin temperature ST meanwhile begins at 34° C. and steadily drops to under 30° C. during the session. The mean body temperature MBT remains slowly and steadily drops during the session, dropping from under 36° C. to approx. 35° C. The mean body temperature MBT drops as a result of the skin temperature ST falling sharply. The heat strain index HSI during the outdoor session is determined to be 0, and therefore the outdoor session does not contribute to the heat strain score HSS.

During the indoor session, which is longer than the outdoor session, the core body temperature CBT rises from under 38° C. to over 39° C. until dropping back down towards 38° C. towards the end of the indoor session. The skin temperature ST rises from 34° C. to over 36° C. before dropping to below 34° C. at the end of the indoor session. The calculated mean body temperature MBT rises from approximately 36° C. to over 38° C. during the session before dropping back to just over 36° C. The heat strain index HSI rises sharply from 0 at the beginning of the session to just under 9 at the time point where the mean body temperature MBT (and correspondingly the core body temperature CBT and the skin temperature ST) are at a maximum. The heat strain index HSI then drops back to 0 before the end of the indoor session. As the heat strain index HSI was non-zero during the indoor session, the heat strain score HSS calculated using the heat strain index HSI was 752.70 during the indoor session, and therefore also 752.70 for the combination of the outdoor session and the indoor session.

The above-described embodiments of the disclosure are exemplary and the person skilled in the art knows that at least some of the components and/or steps described in the embodiments above may be rearranged, omitted, or introduced into other embodiments without deviating from the scope of the present disclosure.

1. A computer-implemented method for determining a measure of the physiological strain on a mammal due to heat, the method comprising:
receiving (S13) a measurement of a core body temperature (CBT) and a skin temperature (ST) of the mammal;
calculating (S14) a mean body temperature (MBT) as a function of the core body temperature (CBT) and the skin temperature (ST); and
calculating (S15) a heat strain index (HSI) as a function of the mean body temperature (MBT), the heat strain index (HSI) indicative of the physiological strain on the mammal due to heat.

2. The method according to claim 1, wherein the mammal is a human and the measurement of the skin temperature is taken at a defined position on the human body, in particular a single defined position, the method comprising correcting the skin temperature (ST) using a defined correction term associated with the defined position.

3. The method according to claim 2, wherein the defined correction term associated with the defined position is determined by the following steps:
measuring the skin temperature at a plurality of different positions on the body, preferably at least three, including the defined position;
calculating a mean skin temperature using the plurality of measurements; and
determining the defined correction term using a difference between the mean skin temperature and the skin temperature at the defined position.

4. The method according to claim 1, the method comprising:
receiving one or more further physiological signals of the mammal, including one or more of: heart rate, heart rate variability, galvanic skin response, sweat rate, breathing rate, oxygen saturation, heat flux, movement data, glucose concentration, or lactate concentration; and
calculating the heat strain index (HSI) further using the one or more further physiological signals.

5. The method according to claim 4, the method comprising calculating the heat strain index (HSI) as a function of the mean body temperature (MBT), a lower temperature threshold (LTT) and a upper temperature threshold (UTT).

6. The method according to claim 1, wherein the core body temperature (CBT) is calculated as a function of a heat flux (HF) at the skin and the skin temperature (ST).

7. The method according to claim 1, wherein the mean body temperature (MBT) is calculated as a linear combination of the core body temperature (CBT) and the skin temperature (ST), preferably a weighted sum comprising a core body temperature coefficient and a skin temperature coefficient.

8. The method according to claim 1, wherein the heat strain index (HSI) is proportional to a difference between the mean body temperature (MBT) and the lower temperature threshold (LTT) and inversely proportional to a difference between the upper temperature threshold (UTT) and the lower temperature threshold (LTT).

9. The method according to claim 1, wherein the method further comprises determining a normalized heat strain index (nHSI) for a period of elevated heat or a part thereof by calculating an i-th power of each of a plurality of heat strain index values, calculating a mean value of the i-th powers, and calculating the i-th root of the mean value, wherein i is a number between 2-5.

10. The method according to claim 1, wherein the method further comprises determining (S17) a heat strain score (HSS) indicative of a cumulative heat load for a period of elevated heat or part thereof, the heat strain score (HSS) determined using the normalized heat strain index (nHSI) and a duration (P) of the period of elevated heat or part thereof.

11. The method according to claim 1, wherein the method further comprises generating an acute heat load (AHL) with reference to a particular time-point as a weighted sum of one or more heat strain scores (HSS) associated with one or more past periods of elevated heat, wherein the weight of a given heat strain score (HSS) depends on a time interval between the particular time-point and the date of the given past period of elevated heat, wherein the weight decreases as the duration increases.

12. The method according to claim 11, wherein the method further comprises generating a chronic heat load (CHL) with reference to a particular time-point as a weighted sum of one or more heat strain scores (HSS) associated with one or more past periods of elevated heat, wherein the weight of a given heat strain score (HSS) depends on a time interval between the particular time-point and the date of the given past period of elevated heat, wherein the weight decreases as the duration increases, wherein the decrease is less rapid than a decrease of the weights used for generating the acute heat load (AHL).

13. The method according to claim 12, further comprising calculating a heat readiness index (HSB) as a function of the chronic heat load (CHL) and the acute heat load (AHL), preferably as a difference between the chronic heat load (CHL) and the acute heat load (AHL).

14. The method according to claim 1, wherein the method further comprises generating an alarm signal if the heat strain index (HSI) exceeds a heat strain index threshold and/or if the heat strain score (HSS) exceeds a heat strain score threshold.

15. The method according to claim 1, wherein the method further comprises generating (S16) a message including the heat strain index (HSI).

16. The method according to claim 1 wherein the method further comprises determining an acclimatization adjusted heat strain index (aaHSI), using the heat strain index (HSI) and one or more further physiological signals of the mammal including one or more of: heart rate, heart rate variability, galvanic skin response, hydration status, body weight, sweat rate, breathing rate, oxygen saturation, heat flux, movement data, performance data, glucose concentration, or lactate concentration.

17. The method according to claim 16, wherein the method further comprises determining an acclimatization index (ACI), indicative of a degree of acclimatization of the person, the acclimatization index determined as a function of the acclimatization adjusted heat strain index (aaHSI) and the heat strain index (HSI).

18. An electronic system (1) for determining a measure of the physiological strain on a mammal due to heat, the electronic system (1) comprising a processor (11) configured to perform the method according to claim 1 one of claims 1 to 17.

19. The electronic system (1) according to claim 18, wherein the electronic system (1) further comprises:
a wearable device (2) including a sensor system (3) configured to determine the core body temperature (CBT) and the skin temperature (ST) of the mammal, wherein the sensor system (3) is worn on the body of the mammal.

20. The electronic system (1) according to claim 19, wherein the sensor system (3) comprises a heat flux sensor (31) configured to determine the core body temperature (CBT) and a temperature sensor (32) configured to determine the skin temperature (ST), in particular the heat flux sensor (31) comprises a series of p- and n-doped semiconductors.

21. The electronic system (1) according to claim 19, comprising one or more of the following sensors: a PPG sensor (34), an ECG sensor (35), a blood lactate sensor, a galvanic skin response sensor, a sweat rate sensor, one or more further skin temperature sensors, an ambient temperature sensor (33), or an accelerometer, preferably the additional sensors are integrated into the wearable device (2).

22. The electronic system (1) according to claim 19, wherein the wearable device (2) comprises a strap for securing the wearable device (2) to the body of a human (8), in particular a chest strap or a wrist strap.

23. The electronic system (1) according to claim 19, wherein the processor (11) is integrated into the wearable device (2).

24. The electronic system (1) according to claim 19, wherein the wearable device (2) includes a wireless communications module configured to generate and transmit a message comprising the current heat strain index (HSI).

25. The electronic system (1) according to claim 19, further comprising a separate electronic device (4, 6), the processor (11) integrated into the separate electronic device (4, 6); the wearable device (2) including a wireless communications module configured to transmit a message including the core body temperature (CBT) and the skin temperature (ST) to the separate electronic device (4, 6).

26. A computer program product for determining a measure of the physiological strain on a mammal due to heat, comprising computer program code configured to control a processor (11) such that the processor (11) performs the method according to claim 1.
